(19) United States
(12) Patent Application Publication
(10) Pub. No.: US 2022/0233796 A1
(43) Pub. Date: Jul. 28, 2022

(54) METHOD FOR CHARGING A CONTAINER FOR USE WITH A MEDICATION DELIVERY APPARATUS, CONTAINER FOR SUCH AN APPARATUS AND METHOD FOR TREATING A PATIENT

(71) Applicant: Mexichem Fluor S.A. de C.V., San Luis Potosi (MX)

(72) Inventors: Stuart Corr, Runcorn (GB); Simon Gardner, Runcorn, Cheshire (GB)

(21) Appl. No.: 17/617,853

(22) PCT Filed: Jun. 8, 2020
(86) PCT No.: PCT/GB2020/051386

§ 371 Date: Dec. 9, 2021

(30) Foreign Application Priority Data
Jun. 11, 2019 (GB) 1908348.4

(51) Int. Cl. A61M 15/00; B65B 3/00; B65B 31/00

(52) U.S. Cl. CPC A61M 15/009; B65B 3/003; C09K 3/30; A61M 2209/045; B65B 31/003

(57) ABSTRACT

A method of charging a container for use in a medication delivery apparatus wherein the propellant used comprises 1,1-difluoroethane (R-152a) is described.

The present invention relates to a method of charging a container for use in a medication delivery apparatus, especially a pressurised aerosol canister for use in a metered dose inhaler (MDI), wherein the propellant used comprises 1,1-difluoroethane (R-152a).

MDIs are the most significant type of inhalation drug delivery system and are well known to those skilled in the art. They are designed to deliver, on demand, a discrete and accurate amount of a drug to the respiratory tract of a patient using liquefied propellant in which the drug is dissolved, suspended or dispersed. The design and operation of MD's is described in many standard textbooks and in the patent literature. However, they all comprise a pressurised container that holds the drug formulation, a nozzle and a valve assembly that is capable of dispensing a controlled quantity of the drug through the nozzle when it is activated. All of these components are typically located in a housing that is equipped with a mouth piece. The drug formulation will comprise a propellant, in which the drug is dissolved, suspended or dispersed, and may contain other materials such as co-solvents, surfactants and preservatives.

In order for a propellant to function satisfactorily in MDIs, it needs to have a number of properties. These include an appropriate boiling point and vapour pressure so that it can be liquefied in a closed container at room temperature but develop a high enough pressure when the MDI is activated to deliver the drug as an atomised formulation even at low ambient temperatures. Further, the propellant should be of low acute and chronic toxicity and have a high cardiac sensitisation threshold. It should have a degree of chemical stability in contact with the drug, the container and the metallic and non-metallic components of the MDI device, and have a low propensity to extract low molecular weight substances from any elastomeric or other polymeric materials in the MDI device. The propellant should also be capable of maintaining the drug in a homogeneous solution, in a stable suspension or in a stable dispersion for a sufficient time. When the drug is in suspension in the propellant, the density of the liquid propellant is desirably similar to that of the solid drug in order to avoid rapid sinking or floating of the drug particles in the liquid. Finally, the propellant should not present a significant flammability risk to the patient in use. In particular, it should form a non-flammable or low flammability mixture when mixed with air in the respiratory tract.

Dichlorodifluoromethane (R-12) possesses a suitable combination of properties and was for many years the most widely used MDI propellant, often blended with trichlorofluoromethane (R-11). Due to international concern that fully and partially halogenated chlorofluorocarbons (CFCs), such as dichlorodifluoromethane and trichlorofluoromethane, were damaging to the earth's protective ozone layer, many countries entered into an agreement, the Montreal Protocol, stipulating that their manufacture and use should be severely restricted and eventually phased out completed. Dichlorodifluoromethane and trichlorofluoromethane were phased out for refrigeration use in the 1990's, but are still used, to some extent in the MDI sector as a result of an essential use exemption in the Montreal Protocol.

1,1,1,2-tetrafluoroethane (R-134a) was introduced as a replacement refrigerant and MDI propellant for R-12. 1,1,1,2,3,3,3-heptafluoropropane (R-227ea) was also introduced as a replacement for R-12 in the fire control (e.g. computer suites) and MDI sectors and is sometimes blended with R-134a for these applications.

Although R-134a and R-227ea have low ozone depletion potentials (ODPs), they have global warming potentials (GWPs), 1430 and 3220 respectively, that are now considered to be too high by some regulatory bodies, especially for dispersive uses when they are released into the atmosphere.

1,1-difluoroethane (R-152a) has been suggested as a replacement MDI propellant for R-134a and R-227ea due to its low global warming potential of 124, in addition to having zero ozone depletion potential (ODP). Toxicological evaluations have demonstrated that R-152a has a very low order of acute and chronic inhalation toxicity, with the compound being neither a mutagen, teratogen or carcinogen. Chemical stability studies have revealed that R-152a does not undergo reaction with solvents commonly used in aerosol formulations, is very stable to hydrolysis, and is compatible with several plastics that are typically prone to attack by solvents and propellants. Furthermore, in EP2706987 the inventors found that the use of R-152a as a propellant reduced the amount of ethanol required for dissolving the drug in the pharmaceutical composition compared to the amount that would be needed if R-134a is used as the propellant. Thus, R-152a has a number of advantageous properties that makes it use as a propellant desirable.

However, while neither R-134a or R-227ea are flammable or explosive under atmospheric conditions, R-152a is both flammable and explosive, having a lower explosive limit (LEL) of 3.9 vol % and an upper explosive limit of 16.9%. The flammability and explosive nature of R-152a means that conventional processes used for charging pressurised aerosol containers, especially those used in MDIs, are unsuitable, as is explained below.

Consequently, it is not normally possible to convert existing processes and facilities to using R-152a as the propellant without significant modification.

There are three conventional processes used for charging pressurised aerosol containers for use in MDIs: cold fill; single-stage pressure fill; and two-stage pressure fill.

Cold fill is a method of manufacture in which cold temperatures are used to convert the drug formulation into the liquid phase. The cold fill process begins with creating a homogenous suspension or solution of the active pharmaceutical ingredient (API) with a solvent or carrier that is a liquid at room temperature. In parallel, the bulk propellant, which forms the rest of the formulation, is placed into a pre-chilled bulk-manufacturing/mixing vessel, where the low temperature ensures the propellant is in liquid form. The concentrate is then transferred into the same vessel, followed by mixing of the entire formulation (comprising the propellant, solvent/carrier, and the API).

The next step of the cold filling process is to dispense the formulation into appropriately sized canisters/containers. This is achieved by pumping the formulation from the mixing vessel to a filling head and feeding a predetermined portion of the chilled liquid formulation into an open canister. Subsequently, a valve assembly is placed on top of each canister and then crimped into place. A seal is formed between the top of each canister and an elastomeric component of the valve assembly.

Each completed canister is then checked for weight to ensure the correct amount of formulation is present. Products may then be subjected to a stress test in a water bath to ensure a proper seal has been formed and that there are no gaps through which the propellant may leak. In the cold fill process, the water bath also serves the purpose of warming the aerosol to room temperature. Even so, the formulation in the canister remains a liquid because it is under pressure.

In contrast to cold filling, both the single-stage and two-stage pressure filling processes use pressure instead of low temperature to maintain the propellant in the liquid phase. In these processes, the propellant is held in a pressurised mixing vessel in liquid form, and a drug concentrate may be made in the same way as it is with cold filling, with the API mixed with a solvent or carrier that is liquid at room temperature.

In the single-stage pressure filling process, the API and propellant are mixed and held under pressure in a bulk-manufacturing/mixing vessel. An empty canister is then fed onto the filling table and a valve assembly placed on top and crimped into place. The complete formulation is then driven under pressure into the canister through the valve assembly. In common with the cold-fill process, the unit is checked, weighed, water bathed and submitted for further processing.

In the two-stage pressure filling process, the API or drug concentrate is placed in an open canister. A valve assembly is then placed on top of the canister and crimped into position to form the seal. The propellant is then driven under pressure backwards through the valve assembly and into the canister. Using this method, the mixing of the concentrate occurs in the canister rather than in a bulk-manufacturing vessel. Following this step, the unit is checked, weighed, water bathed and submitted for further processing.

Each of the three conventional methods used for charging pressurised aerosol containers for use in MDIs are unsuitable when using R-152a, due to its flammability and explosive properties.

In existing cold fill and single-stage pressure fill facilities, the presence of a large bulk-manufacturing/mixing vessel in proximity to the line with a rapid-moving filling head is not a major explosion safety issue, due to the non-flammability and non-explosive nature of R-134a and R-227ea. Accordingly, filling lines are often situation in the core of a building or facility.

However, if R-152a were to be used in an existing facility, the risk profile would be unacceptable due to the flammable and explosive properties of R-152a. Consequently, it is difficult to use an existing cold fill or single-stage pressure fill facility with R-152a without significant modification.

In contrast, in the conventional two-stage pressure fill process, the propellant and API (or drug concentrate) are added separately to the canister, and so there is no requirement for a bulk-manufacturing vessel to mix large quantities of these components. Accordingly, existing two-stage pressure fill facilities may be suitable for use with R-152a with little or no modification.

However, in the convention two-stage pressure fill process, the necessity of adding the API or drug concentrate to the canister before a valve assembly is crimped onto the canister means that it can be difficult to evacuate the sealed canister without risking loss of the API through aerosolisation or foaming, or loss of the solvent or carrier through evaporation.

Consequently, in typical processes, no evacuation of the canister is carried out before propellant is added through the valve. This results in the fully charged canister containing air with a partial pressure of approximately one bar. In some territories, such as the USA, it is mandatory to subject the charged cannister to a stress test in a water bath at elevated temperatures. The additional pressure arising from the air present in the canisters can result in failure of the stress test. The presence of air may also compromise the stability of the API and/or propellant in the canister.

There is a need for a process for charging a pressurised aerosol canister for use in a metered dose inhaler (MDI), wherein the propellant used comprises 1,1-difluoroethane (R-152a), which is broadly compatible with existing facilities and equipment.

The present inventors have surprisingly found that by purging the aerosol canister with a fluid comprising a (hydro)halocarbon and/or by evacuating the canister prior to charging with propellant, it is possible to provide canisters comprising a 1,1-difluoroethane (R-152a) propellant which have the required stress performance, and which may be prepared with only minor modifications to existing facilities and equipment.

Accordingly, in a first aspect of the present invention, there is provided a method for charging a container for use with a medication delivery apparatus comprising;
(a) purging the container with a fluid component comprising a (hydro)halocarbon;
(b) introducing into the container a pharmaceutical composition comprising an active pharmaceutical ingredient;
(c) sealing the container; and
(d) introducing into the container a propellant component comprising 1,1-difluoroethane (R-152a);

wherein the steps are carried out in the order (a), (b), (c) then (d); or

wherein the steps are carried out in the order (b), (a), (c) then (d).

Unless otherwise indicated, all technical and scientific terms used herein have the same meaning as commonly understood by one of ordinary skill in the art to which this invention pertains.

All embodiments of the invention and particular features mentioned herein may be taken in isolation or in combination with any other embodiments and/or particular features mentioned herein (hence describing more particular embodiments and particular features as disclosed herein) without departing from the disclosure of the invention.

As used herein, the term “comprises” will take its usual meaning in the art, namely indicating that the component includes but is not limited to the relevant features (i.e. including, among other things). As such, the term “comprises” will include references to the component consisting essentially of the relevant features. As used herein, the term “consists essentially of” will refer to the relevant component being formed of at least 80% (e.g. at least 85%, at least 90%, or at least 95%, such as at least 99%) of the relevant features, according to the relevant measure (e.g. by weight thereof).

In the process of the first aspect of the invention, steps (a) and (b) may be carried out in either order; that is step (a) may be carried out before step (b), or step (b) may be carried out before step (a). When step (b) is carried out before step (a), care must be taken during the purging of the container with the fluid component not to displace the pharmaceutical composition from the container.

Regardless of the order that steps (a) and (b) are carried out in, both steps are carried out before steps (c) and (d), which are carried out with step (c) before step (d). Thus, the order of the four steps is either (a), (b), (c) then (d); or (b), (a), (c) then (d).

In step (a), the container, especially a canister for use with a metered dose inhaler (MDI), is purged with a fluid component comprising a hydrofluorocarbon. For the avoidance of doubt, the term fluid includes vapours and liquids. Typically, the container, containing an original atmosphere, for example of air or nitrogen, is supplied to a purging station where purging takes place.

In some processes, the fluid component is in the form of a vapour, and may, for example, be delivered at approximately ambient pressure by way of a directed nozzle into the body of the container. The delivered vapour purges the container of the original atmosphere.

In some processes, the fluid component is in the form of a liquid. Evaporation of the liquid in the container purges the container of the original atmosphere.

By the term “purge”, it is meant that an appropriate volume of gas or vapour is delivered, either directly or through evaporation of a liquid, to the container, to displace substantially all of the original atmosphere. Thus, after step (a), the container is substantially free of the original atmosphere of, for example, air or nitrogen.

As used herein, references to “substantially” of a component will refer to at least 50% (e.g. at least 75%, at least 80%, at least 85%, or, particularly, at least 90%, such as at least 95%, or, more particularly, at least 99%) of the component, according to the relevant measure (e.g. by weight thereof).

The purging fluid component used in the process of the invention comprises a hydrofluorocarbon. Some fluid components may comprise at least about 95% by weight of a hydrofluorocarbon, such as at least about 96%, at least about 97%, at least about 98%, at least about 99% or at least about 99.9% by weight of a hydrofluorocarbon. Some purging fluid components may consist entirely of a hydrofluorocarbon.

When used herein in relation to a specific value (such as an amount), the term “about” (or similar terms, such as “approximately”) will be understood as indicating that such values may vary by up to 10% (particularly, up to 5%, such as up to 1%) of the value defined. It is contemplated that, at each instance, such terms may be replaced with the notation “±10%”, or the like (or by indicating a variance of a specific amount calculated based on the relevant value). It is also contemplated that, at each instance, such terms may be deleted.

By the term “(hydro)halocarbons”, we are referring to straight-chain or branched compounds that contain halogen atoms, such as fluorine, chlorine, bromine or iodine, and optionally hydrogen atoms in addition to carbon atoms. Thus, the term includes perhalocarbons as well as hydrohalocarbons which contain halogen and hydrogen atoms in addition to carbon.

Some (hydro)halocarbons that may be mentioned include (hydro)fluorocarbons, preferably hydrofluorocarbons, such as C₂₋₁₀ hydrofluorocarbons, for example C₂-C₅ hydrofluorocarbons.

Some hydrofluorocarbons that may be mentioned include hydrofluoroalkanes, such as 1,1,1,2-tetrafluoroethane (R-134a), 1,1,1,2,3,3,3-heptafluoropropane (R-227ea), 1,1-difluoroethane (R-152a), and mixtures thereof. In some processes, the hydrofluoroalkane is 1,1,1,2-tetrafluoroethane (R-134a), In some processes, the hydrofluoroalkane is 1,1,1,2,3,3,3-heptafluoropropane (R-227ea). In some processes, the hydrofluoroalkane is 1,1-difluoroethane (R-152a).

In some processes that may be mentioned, the hydrofluoroalkane is a mixture of 1,1,1,2-tetrafluoroethane (R-134a) and 1,1-difluoroethane (R-152a). Increasing the amount of 1,1,1,2-tetrafluoroethane (R-134a) in the mixture can be used to reduce the flammability of the mixture compared to 1,1-difluoroethane (R-152a) alone. A mixture of reduced flammability may be useful, for example, if the purging step is to be carried out in existing facilities which have a low flammability rating, for instance metered dose inhaler (MDI) canister filling facilities designed for use with non-flammable propellants, such as 1,1,1,2-tetrafluoroethane (R-134a) and/or 1,1,1,2,3,3,3-heptafluoropropane (R-227ea).

Conveniently, mixtures of 1,1,1,2-tetrafluoroethane (R-134a) and 1,1-difluoroethane (R-152a) may contain up to about 90 weight %, such as up to about 80 weight %, up to about 70 weight %, up to about 60 weight %, up to about 50 weight %, up to about 40 weight %, up to about 30 weight %, up to about 20 weight % or up to about 10 weight % of 1,1,1,2-tetrafluoroethane (R-134a) relative to the total amount of 1,1,1,2-tetrafluoroethane (R-134a) and 1,1-difluoroethane (R-152a) in the mixture.

In other processes that may be mentioned, the hydrofluoroalkane is a mixture of 1,1,1,2,3,3,3-heptafluoropropane (R-227ea) and 1,1-difluoroethane (R-152a). Increasing the amount of 1,1,1,2,3,3,3-heptafluoropropane (R-227ea) in the mixture can be used to reduce the flammability of the mixture compared to 1,1-difluoroethane (R-152a) alone. A mixture of reduced flammability may be useful, for example, if the purging step is to be carried out in existing facilities which have a low flammability rating, for instance metered dose inhaler (MDI) canister filling facilities designed for use with non-flammable propellants, such as 1,1,1,2-tetrafluoroethane (R-134a) and/or 1,1,1,2,3,3,3-heptafluoropropane (R-227ea).

Conveniently, mixtures of 1,1,1,2,3,3,3-heptafluoropropane (R-227ea) and 1,1-difluoroethane (R-152a) may contain up to about 90 weight % such as up to about 80 weight %, up to about 70 weight %, up to about 60 weight %, up to about 50 weight %, up to about 40 weight %, up to about 30 weight %, up to about 20 weight % or up to about 10 weight % of 1,1,1,2,3,3,3-heptafluoropropane (R-227ea) relative to the total amount of 1,1,1,2,3,3,3,-heptafluoropropane (R-227ea) and 1,1-difluoroethane (R-152a) in the mixture.

In other processes that may be mentioned, the hydrofluoroalkane is a mixture of 1,1,1,2-tetrafluoroethane (R-134a) and 1,1,1,2,3,3,3-heptafluoropropane (R-227ea). Conveniently, mixtures of 1,1,1,2-tetrafluoroethane (R-134a) and 1,1,1,2,3,3,3-heptafluoropropane (R-227ea) may contain up to about 90 weight %, such as up to about 80 weight %, up to about 70 weight %, up to about 60 weight %, up to about 50 weight %, up to about 40 weight %, up to about 30 weight %, up to about 20 weight % or up to about 10 weight % of 1,1,1,2-tetrafluoroethane (R-134a) relative to the total amount of 1,1,1,2-tetrafluoroethane (R-134a) and 1,1,1,2,3,3,3-heptafluoropropane (R-227ea) in the mixture,

Some other hydrofluorocarbons that may be mentioned include hydrofluoroolefins such as hydrofluoropropenes. Some hydrofluoropropenes that may be mentioned include tetrafluoropropenes, such as 1,3,3,3-tetrafluoropropene (R-1234ze) and 2,3,3,3-tetrafluoropropene (R-1234yf), preferably 1,3,3,3-tetrafluoropropene (R-1234ze). 1,3,3,3-tetrafluoropropene (R-1234ze) is available as two geometric isomers, trans-1,3,3,3-tetrafluoropropene (R-1234ze(E)) and cis-1,3,3,3-tetrafluoropropene (R-1234ze(Z)), of which trans-1,3,3,3-tetrafluororpropene (R-1234ze(E)) is preferred.

Where the purging fluid component used in step (a) is flammable, the purging station and adjacent equipment is suitably designed to mitigate the risks associated with the relative small volumes of flammable fluid component used.

In step (b), a pharmaceutical composition comprising an active pharmaceutical ingredient is introduced into the container. In a metered dose inhaler (MDI) canister facility, the canister is supplied to a charging station where the pharmaceutical formulation is metered into the canister.

The active pharmaceutical ingredient in the pharmaceutical composition may comprise one or more pharmaceutical substances that are suitable for delivery through an oral or nasal aerosol delivery route. Relevant pharmaceutical substances include corticosteroids (ICS); short acting beta-2-agonists (SABA); long acting beta-2-agonists (LABA); long acting muscarinic antagonists (LAMA); short acting muscarinic antagonists (SAMA); cromoglicate (for example sodium cromoglicate); synthetic, semi-synthetic or natural cannabinoids; synthetic, semi-synthetic or natural opioids; or combinations thereof. Other relevant pharmaceutical substances include nicotine. The active pharmaceutical ingredient may comprise a combination of substances from the above-described classes of pharmaceutical substances.

The active pharmaceutical ingredient may also be used in combination with one or more excipients including solvents, co-solvents, co-suspension agents and surfactants.

In some methods that may be mentioned, the active pharmaceutical ingredient comprises or consists of a corticosteroid. Any of the corticosteroids that that are suitable for delivery through an oral or nasal aerosol delivery route, such as those that have been in use hitherto for treating asthma and chronic obstructive pulmonary diseases and that can be delivered using a MDI, can be used in the methods of the present invention. Suitable corticosteroids include budesonide, mometasone, beclomethasone and fluticasone as well as their pharmaceutically acceptable derivatives, such as their pharmaceutically acceptable salts and esters. Preferred compounds include budesonide, mometasone furoate, beclomethasone dipropionate and fluticasone propionate. The most preferred corticosteroids are budesonide, mometasone, fluticasone and beclomethasone, particularly budesonide and mometasone and especially budesonide.

In some methods that may be mentioned, the active pharmaceutical ingredient comprises or consists of a short acting beta-2-agonist (SABA). Any of the short acting beta-2-agonists that are suitable for delivery through an oral or nasal aerosol delivery route, such as those that have been in use hitherto for treating asthma and chronic obstructive pulmonary diseases and that can be delivered using a MDI, can be used in the methods of the present invention. Suitable short acting beta-2-agonists include levosalbutamol, salbutamol and terbutaline as well as their pharmaceutically acceptable derivatives, such as their pharmaceutically acceptable salts and esters. Preferred compounds include salbutamol and salbutamol sulphate.

In some methods that may be mentioned, the active pharmaceutical ingredient comprises or consists of a long acting beta-2-agonist (LABA). Any of the long acting beta-2-agonists that that are suitable for delivery through an oral or nasal aerosol delivery route, such as those that have been in use hitherto for treating asthma and chronic obstructive pulmonary diseases and that can be delivered using a MDI, can be used in the methods of the present invention. Suitable long acting beta-2-agonists include formoterol, arformoterol, bambuterol, clenbuterol, salmeterol, indacaterol and olodaterol as well as their pharmaceutically acceptable derivatives, such as their pharmaceutically acceptable salts and esters. Preferred compounds include formoterol, salmeterol and olodaterol and the pharmaceutically acceptable salts thereof. Particularly preferred compounds include formoterol fumarate, formoterol fumarate dihydrate, salmeterol xinafoate and oladaterol.

In some methods that may be mentioned, the active pharmaceutical ingredient comprises or consists of a long acting muscarinic antagonist (LAMA). Any of the long acting muscarinic antagonists that that are suitable for delivery through an oral or nasal aerosol delivery route, such as those that have been in use hitherto for treating asthma and chronic obstructive pulmonary diseases and that can be delivered using a MDI, can be used in the methods of the present invention. Suitable long acting muscarinic antagonists include ipratropium, tiotropium, aclidinium and the pharmaceutically acceptable derivatives thereof, especially the pharmaceutically acceptable salts thereof. Preferred compounds include the pharmaceutically acceptable salts of glycopyrrolate (also known as glycopyrronium). Glycopyrrolate is a quaternary ammonium salt. Suitable pharmaceutically acceptable counter ions include, for example, fluoride, chloride, bromide, iodide, nitrate, sulfate, phosphate, formate, acetate, trifluoroacetate, propionate, butyrate, lactate, citrate, tartrate, malate, maleate, succinate, benzoate, p-chlorobenzoate, diphenyl-acetate or triphenylacetate, o-hydroxybenzoate, p-hydroxybenzoate, 1-hydroxynaphthalene-2-carboxylate, 3-hydroxynaphthalene-2-carboxylate, methanesulfonate and benzenesulfonate. A preferred compound is the bromide salt of glycopyrrolate also known as glycopyrronium bromide.

In some methods that may be mentioned, the active pharmaceutical ingredient comprises or consists of synthetic or natural cannabinoids. Any of the cannabinoids that that are suitable for delivery through an oral or nasal aerosol delivery route, such as those that have been in use hitherto for treating pain, seizures, arthritis, nausea, neurodegenerative diseases, such as multiple sclerosis, cancer and HIV, or for treating asthma and chronic obstructive pulmonary diseases, and that can be delivered using a MDI, can be used in the methods of the present invention. Suitable cannabinoids include tetrahydrocannabinols (THC), such as delta-9-tetrahydrocannabinol, delta-8-tetrahydrocannabinol and cannabidiol (CBD).

In some methods that may be mentioned, the active pharmaceutical ingredient comprises or consists of synthetic, semi-synthetic or natural opioids. Any of opioids that that are suitable for delivery through an oral or nasal aerosol delivery route can be used in the methods of the present invention. Suitable opioids include morphine or methadone. In other methods that may be mentioned, the active pharmaceutical ingredient comprises a combination of a cannabinoid and an opioid.

The pharmaceutical composition may be in the form of a solid, a solution or a suspension. Some pharmaceutical compositions may be in the form of a pelletised solid. When the pharmaceutical composition is in the form of a solid, it is particularly preferred that the solid is pelletised. Equipment for pellet formation of pharmaceutical products is common in the field. The particle size and cohesive strength of the pelletized solid should be large enough to resist aerosolisation of the pharmaceutical composition when the sealed container is evacuated (in step (iii)), but small enough to still permit good dispersion of the composition in the propellant. The pharmaceutical pellet may include excipients to optimise the mechanical or dispersive properties of the pellet.

In addition to the active pharmaceutical ingredient, the pharmaceutical composition may comprise a number of additional components. These components may be present in the pharmaceutical composition before it is added to the container. Alternatively, the components may be added to the container separately from the pharmaceutical composition, such as before or after the pharmaceutical composition is added to the container.

Such additional components may include a carrier solvent in which the active pharmaceutical ingredient is soluble, for example, ethanol.

Such additional components may include a surfactant, which produces a more stable suspension. Commonly used surfactants include oleic acid, lecithin, sorbitan trioleate, polyvinylpyrrolidone and polyethylene glycol.

The pharmaceutical compositions may also comprise one or more other additives of the type that are conventionally used in drug formulations for metered dose inhalers (MDIs), such as valve lubricants. Where other additives are included in the pharmaceutical composition, they are normally used in amounts that are conventional in the art.

The container may be filled with enough of the pharmaceutical composition to provide for a plurality of dosages. The pressurised aerosol canisters that are used in MDIs typically contain 50 to 200 individual doses.

In step (c), the container is sealed. By sealing, it is meant that the open portion of the container is closed, covered or obstructed to prevent substantial loss of the fluid component or ingress of ambient atmosphere.

It is preferable that step (c) is carried out before substantially any displacement of the fluid component (introduced in step (a) from the container) by ambient atmosphere (such as air or nitrogen) occurs, otherwise the benefit achieved by step (a) will be reduced. Consequently, the stations at which steps (a) to (c) are carried out are ideally located adjacent to each other on a filling production line such that the time between purging, introducing the pharmaceutical composition, and sealing of the container is minimised. Alternatively or additionally, steps (a) to (c) may be carried out in an atmosphere of the fluid component, such that there is no ingress of air or nitrogen into the container during steps (b) and (c), or in the event that there is a significant delay between carrying step (a) and either of steps (b) or (c).

In some processes that may be mentioned, the unsealed container, such as an unsealed canister for use in a metered dose inhaler (MDI), does not contain a valve. Such containers may be sealed by affixing (for example, by crimping) a cap comprising a valve over the open portion of the container. The presence of a valve in the sealed canister permits the introduction of the propellant composition in step (d), and, ultimately, allows the dispensing of a metered dose of the pharmaceutical composition by the end user. The cap may also comprise other elements necessary for the functioning of the container in the medication delivery apparatus.

In some processes that may be mentioned, the unsealed container may already contain a valve and any other elements necessary for the introduction of the propellent composition in step (d) and, ultimately, to allow the dispensing of a metered dose of the pharmaceutical composition by the end user when the container is fitted to the medication delivery apparatus. In these processes, the container may be sealed through affixing a cap, for example a monolithic cap, over the open portion of the container. Alternatively, some containers may be sealed without the use of an additional element, for example where the open portion of the container may be crimped closed.

In step (d), a propellant composition comprising 1,1-difluoroethane (R-152a) is introduced into the container. Typically, the sealed container, containing the pharmaceutical composition and the fluid component at atmospheric pressure, is supplied to a propellant charging station where the liquefied propellant composition, under pressure, is metered into the container through the valve. In some processes, the propellant charging station is located at a location remote from the stations for steps (a), (b) and (c), where the flammability and explosive hazards associated with handling flammable liquid propellant have been appropriately mitigated.

Some propellant compositions that may be mentioned comprise at least about 95% by weight of 1,1-difluoroethane (R-152a), such as at least about 96%, at least about 97%, at least about 98%, at least about 99% or at least about 99.9% by weight of 1,1-difluoroethane (R-152a). Some propellant compositions consist entirely of 1,1-difluoroethane (R-152a).

In some processes that may be mentioned, the absolute pressure in the sealed and charged container (i.e. after step (d) has been completed) at 293K is within the range of about 400 kPa to about 600 kPa, preferably about 450 kPa to about 600 kPa, more preferably about 500 kPa to about 600 kPa, even more preferably about 500 kPa to about 550 kPa, most preferably about 500 kPa to about 520 kPa. In preferred embodiments of the invention, these pressure ranges apply when between about 50% and about 75% of the volume of the container is occupied by the liquid component of the propellant composition, such as wherein between about 55% and 70% of the volume of the container is occupied by the liquid component of the propellant composition, for example wherein between about 60% and 65% of the volume of the container is occupied by the liquid component of the propellant composition. In preferred embodiments of the invention, these pressure ranges apply when the propellant composition comprises at least about 95% by weight of 1,1-difluoroethane (R-152a), such as at least about 96%, at least about 97%, at least about 98%, at least about 99% or at least about 99.9° k by weight of 1,1-difluoroethane (R-152a), for example consisting essentially of 1,1-difluoroethane (R-152a).

Subsequent to step (d), the charged container may be conveyed to other stations to be equipped with additional device components such as actuators and dose counters, to be labelled, to be packaged and to be warehoused. The container may also be sonicated or otherwise subject to mechanical agitation to ensure dissolution or uniform dispersion of the pharmaceutical composition in the propellant.

In some processes that may be mentioned, the charged container is subjected to an integrity/stress and/or leak test which comprises the step of submerging the container in a liquid at a temperature of about 30 to about 80° C., such as about 40 to about 70° C., for example about 50 to about 60° C., or about 55° C. for a period of about 1 to about 5 minutes, such as about 2 to about 4 minutes, for example about 3 minutes. The integrity and/or leak test may be carried out in a water bath.

In some processes of the invention, the container is a pressured aerosol canister for use with a metered dose inhaler (MDI).

Without wishing to be bound by theory, it is believed that the purging of the container with a fluid component reduces the amount of ambient atmosphere present in the sealed container, which reduces the pressure in the container during integrity/stress testing at elevated temperatures. It is believed that the reduced pressure results from the lower saturated vapour pressure of the fluid component compared to ambient atmosphere, which predominantly comprises nitrogen.

According to a second aspect of the present invention, there is provided a method for charging a container for use with a medical delivery apparatus comprising:
(i) introducing into the container a pharmaceutical composition comprising an active pharmaceutical ingredient;
(ii) sealing the container;
(iii) optionally, at least partially evacuating the container;
(iv) optionally, introducing into the container a fluid component comprising a (hydro)halocarbon; and
(v) introducing into the container a propellant component comprising 1-difluoroethane (R-152a);

wherein steps (i) to (v) are carried out in the stated order; and

wherein at least one of steps (iii) and (iv) are mandatory.

In step (i), a pharmaceutical composition comprising an active pharmaceutical ingredient is introduced into the container in the same ways as described in relation to step (b) of the first aspect of the invention. In particular, all embodiments relating to step (b) of the first aspect of the invention and all features described therein also apply to step (i) of the second aspect of the invention.

When the pharmaceutical composition is in the form of a solid, it is particularly preferred that the solid is pelletised. Equipment for pellet formation of pharmaceutical products is common in the field. The particle size and cohesive strength of the pelletized solid should be large enough to resist aerosolisation of the pharmaceutical composition when the sealed container is evacuated (in step (iii)), but small enough to still permit good dispersion of the composition in the propellant. The pharmaceutical pellet may include excipients to optimise the mechanical or dispersive properties of the pellet.

In step (ii), the container is sealed in the same ways as described in relation to step (c) of the first aspect of the invention. In particular, all embodiments relating to step (c) of the first aspect of the invention and all the features described therein also apply to step (ii) of the second aspect of the invention.

Step (iii) is an optional step. In step (iii), the sealed container is at least partially evacuated. As used herein, by the term “evacuated”, it is meant that substantially all of the atmosphere in the container is removed, typically through the valve. Evacuation of the container may be carried out by any means known in the art, for example, using a vacuum pump.

Step (iv) is an optional step. When both steps (iii) and (iv) are carried out, in step (iv) a fluid component is introduced into the at least partially evacuated container. The fluid component may be introduced into the container through the valve at ambient pressure. Since many of the valve assemblies for canisters used with metered dose inhalers (MDIs) are intended to provide a sealing function at elevated internal pressures due to the presence of the propellant, the introduction of the fluid component into the evacuated container reduces the potential for subsequent ingress of ambient atmosphere, such as air, into the sealed container prior to propellant charging (i.e. step (v)).

When step (iii) is not carried out, in step (iv) a fluid component is introduced into the container to purge the container of the original atmosphere. The fluid component may be introduced, and the original atmosphere displaced through the same valve, or through different valves.

All embodiments relating to step (a) of the first aspect of the invention and all the features described therein apply to step (iv) of the second aspect of the invention.

At least one of steps (iii) and (iv) is mandatory. In some embodiments, step (iii) is carried out and step (iv) is not carried out. In other embodiments, step (iii) is not carried out and step (iv) is carried out. In yet further embodiments, both steps (iii) and (iv) are carried out.

In step (v), a propellant component comprising 1,1-difluoroethane (R-152a) is introduced into the container in the same ways as described in relation to step (d) of the first aspect of the invention. In particular, all embodiments relating to step (d) of the first aspect of the invention and all features described therein also apply to step (v) of the second aspect of the invention.

According to a third aspect of the invention, there is provided a container for a medication delivery apparatus produced by the method of the first or second aspects of the invention (including all embodiments and/or particular features mentioned therein).

According to a fourth aspect of the invention, there is provided a medication delivery apparatus fitted with a container of the third aspect of the invention (including all embodiments and/or particular features mentioned therein). Preferably, the medication delivery apparatus is a metered dose inhaler (MDI) and the container is a pressurised aerosol canister for use with a metered dose inhaler (MDI).

The pharmaceutical compositions present in the containers produced by the methods of the present invention are for use in medicine for treating a patient suffering or likely to suffer from a respiratory disorder and especially asthma or a chronic obstructive pulmonary disease.

Accordingly, in a fifth aspect of the invention, there is provided a method for treating a patient suffering or likely to suffer from a respiratory disorder which comprises administering to the patient a therapeutically or prophylactically effective amount of the pharmaceutical composition from the container of the third aspect of the invention (including all embodiments and/or particular features mentioned therein). The pharmaceutical composition is preferably delivered to the patient using an MDI of the fourth aspect of the invention (including all embodiments and/or particular features mentioned therein).

Without wishing to be bound by theory, it is believed that the evacuation of the container before introduction of the propellant component reduces the internal pressure of the container during integrity/stress testing at elevated temperatures. Furthermore, the evacuation of the container followed by the introduction of the fluid component reduces the potential for subsequent ingress of ambient atmosphere, such as air, into the sealed container prior to propellant charging, while reducing the pressure in the container during integrity/stress testing at elevated temperatures. It is believed that the reduced pressure results from the lower saturated vapour pressure of the fluid component compared to ambient atmosphere, which predominantly comprises nitrogen.

1. A method for charging a container for use with a medication delivery apparatus comprising:
(a) purging the container with a fluid component comprising a (hydro)halocarbon;
(b) introducing into the container a pharmaceutical composition comprising an active pharmaceutical ingredient;
(c) sealing the container; and
(d) introducing into the container a propellant component comprising 1,1-difluoroethane (R-152a);
wherein the steps are carried out in the order (a), (b), (c) then (d); or
wherein the steps are carried out in the order (b), (a), (c) then (d).

2. The method of claim 1, wherein the steps are carried out in the order (a), (b), (c) then (d) or in the order (b), (a), (c) then (d).

3. (canceled)

4. The method of claim 1, wherein the fluid component is a vapour or a liquid.

5. (canceled)

6. The method of claim 1, wherein step (c) is carried out before substantially any displacement of the fluid component from the container by ambient atmosphere occurs.

7. A method for charging a container for use with a medication delivery apparatus comprising:
(i) introducing into the container a pharmaceutical composition comprising an active pharmaceutical ingredient;
(ii) sealing the container;
(iii) optionally, at least partially evacuating the container;
(iv) optionally, introducing into the container a fluid component comprising a hydrofluorocarbon; and
(v) introducing into the container a propellant component comprising 1,1-difluoroethane (R-152a);
wherein steps (i) to (v) are carried out in the stated order; and
wherein at least one of steps (iii) and (iv) are mandatory.

8. The method of claim 7, wherein at least about 95 weight percent of the fluid component is (hydro)halocarbon,
preferably wherein at least 99 weight percent of the fluid component is (hydro)halocarbon;
more preferably wherein at least 99.9 weight percent of the fluid component is (hydro)halocarbon;
even more preferably wherein the fluid component is entirely (hydro)halocarbon;
based on the total weight of the fluid component.

9. The method of claim 7, wherein the (hydro)halocarbon is a hydrofluoroalkane or a hydrofluoralkane selected from the group consisting of 1,1,1,2-tetrafluoroethane (R-134a), 1,1,1,2,3,3,3-heptafluoropropane (R-227ea), 1,1-difluoroethane (R-152a), and mixtures thereof.

10. (canceled)

11. The method of claim 7, wherein the hydrofluoroalkane is a mixture of 1,1,1,2-tetrafluorethane (R-134a) and 1,1-difluoroethane (R-152a) or a mixture of 1,1,1,2,3,3,3-heptafluoropropane (R-227ea) and 1,1-difluoroethane (R-152a) or a mixture of 1,1,1,2-tetrafluoroethane (R-134a) and 1,1,1,2,3,3,3-heptafluoropropane (R-227ea).

12.-13. (canceled)

14. The method of claim 1, wherein the hydrofluorocarbon is a hydrofluoroolefin.

15. The method of claim 14, wherein the hydrofluorolefin is a tetrafluoropropene such as 1,3,3,3-tetrafluoropropene (R-1234ze), preferably trans-1,3,3,3-tetrafluoropropene (R-1234ze(E)).

16. (canceled)

17. The method of claim 1, wherein the pharmaceutical composition is in the form of a solid, a solution or a suspension.

18. The method of claim 17, wherein the pharmaceutical composition is in the form of a solid, such as a pelletised solid.

19. The method of claim 1, wherein the step of sealing the container comprises affixing a cap comprising a valve over the open portion of the container.

20. The method of claim 1, wherein at least about 95 weight percent of the propellant component is 1,1-difluoroethane (R-152a);
preferably at least about 99 weight percent of the propellant component is 1,1-difluoroethane (R-152a);
more preferably at least about 99.9 weight percent of the propellant component is 1,1-difluoroethane (R-152a);
even more preferably wherein the fluid component is entirely 1,1-difluoroethane (R-152a);
based on the total weight of the propellant component.

21. The method of claim 1, wherein the absolute pressure at 293K in the container after the step of introducing into the container a propellant component comprising 1,1-difluoroethane (R-152a) is about 500 kPa to about 600 kPa, preferably about 500 kPa to about 550 kPa, even more preferably about 500 kPa to about 520 kPa.

22. The method of claim 1, which further comprises the step of submerging the container in a liquid at a temperature of about 50 to about 60 degrees centigrade for a period of about 2 to about 4 minutes; such as a temperature of about 55 degrees centigrade for a period of about 3 minutes.

23. The method of claim 1, wherein the active pharmaceutical ingredient comprises a corticosteroid (ICS), a short acting beta-2-agonist (SABA), a long acting beta-2-agonist (LABA), a short acting muscarinic antagonist (SAMA), a long acting muscarinic antagonist (LAMA), a cannabinoid, an opioid, cromoglicate, nicotine, or a combination thereof.

24. (canceled)

25. The method of claim 23, wherein;
the corticosteroid is selected from the group of consisting of budesonide, mometasone, beclomethasone, fluticasone, and pharmaceutically acceptable salts and esters thereof; preferably budesonide, mometasone furcate, beclomethasone dipropionate and fluticasone propionate;
wherein the short acting beta-2-agonist is selected from the group consisting of levosalbutamol, salbutamol, terbutaline, and pharmaceutically acceptable salts and esters thereof; preferably salbutamol and salbutamol sulphate;
the long acting beta-2-agonist is selected from the group consisting of formoterol, arformoterol, bambuterol, clenbuterol, salmeterol, indacaterol, olodaterol, and pharmaceutically acceptable salts and esters thereof; preferably formoterol fumarate, formoterol fumarate dihydrate, salmeterol xinafoate and oladaterol;
the long acting muscarinic antagonist (LAMA) is selected from the group consisting of ipratropium, tiotropium, aclidinium, pharmaceutically acceptable salts and esters thereof, and pharmaceutically acceptable salts of glycopyrrolate; preferably glycopyrronium bromide;
the cannabinoid is a tetrahydrocannabinol (THC), such as delta-9-tetrahydrocannabinol, delta-8-tetrahydrocannabinol or cannabidiol (CBD); and
the opioid is selected from the group consisting of morphine or methadone.

26.-30. (canceled)

31. The method of claim 1, wherein the container is a pressurised aerosol canister for use with a metered dose inhaler (MDI).

32. A container for a medication delivery apparatus produced by the method of claim 1.

33. A medication delivery apparatus fitted with a container according to claim 32, preferably wherein the medication delivery apparatus is a metered dose inhaler (MDI) and wherein the container is a pressurised aerosol canister for use with a metered dose inhaler (MDI).

34. A method for treating a patient suffering or likely to suffer from a respiratory disorder which comprises administering to the patient a therapeutically or prophylactically effective amount of the active pharmaceutical ingredient from the container of claim 32.
